(19) United States
(12) Patent Application Publication
(10) Pub. No.: US 2015/0065526 A1
(43) Pub. Date: Mar. 5, 2015

(54) OVERCOMING ACQUIRED RESISTANCE TO CHEMOTHERAPY TREATMENTS THROUGH SUPPRESSION OF STAT3

(71) Applicant: EMORY UNIVERSITY, Atlanta, GA (US)

(72) Inventors: Xingming Deng, Lilburn, GA (US); Rui Li, Decatur, GA (US); Fadio R. Khuri, Atlanta, GA (US); Taofeek Kunle Owonikoko, Atlanta, GA (US)

(21) Appl. No.: 14/478,238

(22) Filed: Sep. 5, 2014

Related U.S. Application Data
(60) Provisional application No. 61/873,919, filed on Sep. 5, 2013.

(51) Int. Cl. A61K 31/517; A61K 31/166

(52) U.S. Cl. CPC A61K 31/517; A61K 31/166
USPC 514/266.4

(57) ABSTRACT

This disclosure relates to methods of treating cancer comprising administering an effective amount of a STAT3 inhibitor, prodrugs, or derivatives thereof, in combinations with an EGFR tyrosine kinase inhibitors, prodrugs, or derivatives thereof, to a subject in need thereof. In certain embodiments, the STAT3 inhibitor is niclosamide or salt thereof. In certain embodiments, the EGFR tyrosine kinase inhibitor is erlotinib or salt thereof.

### CROSS-REFERENCE TO RELATED APPLICATIONS

This Application claims priority to U.S. Provisional Application No. 61/873,919 filed Sep. 5, 2013, hereby incorporated by reference in its entirety.

### GOVERNMENT ACKNOWLEDGMENT

This invention was made with government support under Grants R01CA112183 and R01CA136534 awarded by the National Institutes of Health. The Government has certain rights in the invention.

### BACKGROUND

The overall 5-year survival rates for non-small cell lung cancer (NSCLC) and small cell lung cancer (SCLC) are low. The EGF receptor (EGFR) has been established as a therapeutic target for the treatment of NSCLC because a significant proportion harbor somatic mutations in the tyrosine kinase domain of the EGFR gene that promote cancer cell growth. EGFR tyrosine kinase inhibitors (TKI) such as erlotinib or gefitinib have been established as one of the therapeutic options for a well-defined subset of patients with lung cancer. Acquired tumor resistance to these agents typically emerges after 6 to 12 months of therapy and constitutes the major limitation of this otherwise effective therapeutic approach. The mechanisms of this resistance are not fully understood but may be associated with activation of EGFR-independent pathways, occurrence of additional EGFR gene mutations, or loss of the target. For example, the EGFR T790M kinase mutation may account for approximately 50% of the acquired TKI-resistant cases, and MET genomic amplification is correlated with about 5% to 11% of acquired TKI resistance.

STAT3 is a physiologic transcription factor for Bcl2 and Bcl-XL, and erlotinib activation of STAT3 has been previously shown to result in upregulation of Bcl2/Bcl-XL thereby contributing to resistance of lung cancer cells to erlotinib. Fan et al. report disrupting the mitochondrial BCL-2/BCL-XL anti-apoptotic machinery in early survivor cells using BCL-2 Homology Domain 3 (BH3) mimetic agents such as ABT-737, or by dual RNAi-mediated knockdown of BCL-2/BCL-XL, was sufficient to eradicate the early-resistant lung-tumor-cells evading targeted inhibitors. Cancer Res, 2011, 71:4494-505.

STAT3 has been shown to be an important molecule in a variety of malignancies and is a therapeutic target in cancer therapy. The latent cytoplasmic STAT3 becomes activated through phosphorylation at residue Tyr705 by Janus-activated kinase (JAK) or growth factor receptor-associated tyrosine kinase. Phosphorylated STAT3 (pSTAT3) dimerizes through a reciprocal Src homology 2-phospho-tyrosine interaction and accumulates in the nucleus, where it activates the transcription of a wide array of genes, including Bcl2/Bcl-XL, cyclin D1, c-Myc, etc. In addition to STAT3 kinases (i.e., JAK and Src), STAT3 phosphorylation is also tightly regulated by a process of dephosphorylation, which is mediated by the protein tyrosine phosphatase, PTPMeg2. PTPs are a large and structurally diverse family of enzymes that catalyze the dephosphorylation of phosphorylated proteins. PTPMeg2 has recently been identified as a physiologic STAT3 phosphatase that directly dephosphorylates STAT3 at the Tyr705 residue.

Ren et al. report niclosamide as an inhibitor of the STAT3 signaling pathway. Acs Med Chem Lett, 2010, 1:454-9.

Sack et al. report an effect of antihelminthic niclosamide on S100A4-mediated metastatic progression in colon cancer. J Natl Cancer Inst, 2011, 103:1018-36

Fan et al. report MET-independent lung cancer cells evading EGFR kinase inhibitors are therapeutically susceptible to BH3 mimetic agents. Cancer Res, 2011, 71:4494-505

References cited herein are not an admission of prior art.

### SUMMARY

This disclosure relates to methods of treating cancer comprising administering an effective amount of a STAT3 inhibitor, prodrugs, or derivatives thereof, in combinations with an EGFR tyrosine kinase inhibitors, prodrugs, or derivatives thereof, to a subject in need thereof. In certain embodiments, the STAT3 inhibitor is niclosamide or salt thereof. In certain embodiments, the EGFR tyrosine kinase inhibitor is erlotinib or salt thereof.

In certain embodiments, the subject is diagnosed with leukemia, melanoma, cervical, ovarian, colon, breast, gastric, lung, skin, ovarian, pancreatic, prostate, head, neck, and renal cancer.

In certain embodiments, the subject is diagnosed with head cancer, neck cancer, pancreatic cancer, small cell lung cancer or non-small cell lung cancer. In certain embodiments, the subject is administered niclosamide at 100 to 5,000 mg per day, or 500 to 3,000 mg per day and the subject is administered erlotinib at 50 to 500 mg or 100 to 200 mg per day.

In certain embodiments, the disclosure contemplates pharmaceutical compositions comprising a STAT3 inhibitor, prodrugs, or derivatives thereof, in combinations with an EGFR tyrosine kinase inhibitor, prodrugs, or derivatives thereof and a pharmaceutically acceptable excipient.

In certain embodiments, the STAT3 inhibitor is selected from cucurbitacin I, auranofin, galiellalactone, kahweol, cepharanthine, nifuroxazide, cryptotanshinone and static.

In certain embodiments, the agents are administered in combination with another chemotherapeutic agent such as, but not limited to, docetaxel, cis-platin, 5-fluorouracil, gemcitabine, tegafur, raltitrexed, methotrexate, cytosine arabinoside, hydroxyurea, adriamycin, bleomycin, doxorubicin, daunomycin, epirubicin, idarubicin, mitomycin-C, dactinomycin and mithramycin, vincristine, vinblastine, vindesine, vinorelbine taxol, taxotere, etoposide, teniposide, amsacrine, topotecan, camptothecin bortezomib anegrilide, tamoxifen, toremifene, raloxifene, droloxifene, iodoxyfene fulvestrant, bicalutamide, flutamide, nilutamide, cyproterone, goserelin, leuprorelin, buserelin, megestrol, anastrozole, letrozole, vorazole, exemestane, finasteride, marimastat, trastuzumab, dasatinib, bevacizumab, combretastatin, thalidomide, and/or lenalidomide or combinations thereof.

In certain embodiments, the EGFR tyrosine kinase inhibitor is gefitinib or salt thereof, and the subject is diagnosed with lung or breast cancer.

In certain embodiments, the EGFR tyrosine kinase inhibitor is imatinib or salt thereof, and the subject is diagnosed with leukemia or chronic myelogenous leukemia.

In certain embodiments, the EGFR tyrosine kinase inhibitor is cetuximab or panitumumab or salt thereof, and the subject is diagnosed with colon or colorectal cancer.

In certain embodiments, the EGFR tyrosine kinase inhibitor is zalutumab or nimotuzumab or salt thereof, and the subject is diagnosed with squamous cell carcinoma of the head and neck (SCCHN).

In certain embodiments, the EGFR tyrosine kinase inhibitor is nimotuzumab or salt thereof, and the subject is diagnosed with glioma.

In certain embodiments, the EGFR tyrosine kinase inhibitor is matuzumab or salt thereof, and the subject is diagnosed with colorectal, lung, esophageal or stomach cancer.

In certain embodiments, the EGFR tyrosine kinase inhibitor is lapatinib or salt thereof, and the subject is diagnosed with HER2-positive breast cancer.

In certain embodiments, the disclosure relates to therapeutic methods disclosed herein wherein the pharmaceutical compositions are administered before, after or during radiotherapy.

In certain embodiments, the disclosure relates to uses of compounds disclosed herein in the production of a medicament for the treatment or prevention of cancer.

### BRIEF DESCRIPTION OF THE FIGURES

FIG. 1 shows data indicating inhibition of EGFR by erlotinib downregulates PTPMeg2 in association with the activation of the STAT3/Bcl2/Bcl-XL pathway in lung cancer HCC827 cells. A, HCC827 cells were treated with erlotinib (Erlo) as indicated. pEGFR, PTPMeg2, pSTAT3, Bcl2, Bcl-XL, etc., were analyzed by Western blot analysis. B, HCC827 cells were treated with erlotinib (0.1 μmol/L) for various times. Levels of Bcl2 or Bcl-XL mRNA were analyzed by RT-PCR.

FIG. 2 shows data indicating depletion of PTPMeg2 from HCC827 cells leads to upregulation of pSTAT3 and Bcl2/Bcl-XL. A, PTPMeg2 shRNA or control shRNA was transfected into HCC827 cells. Expression levels of pSTAT3, Bcl-XL, Bcl2, and Mcl-1 were analyzed by Western blot analysis. B, HCC827 cells expressing PTPMeg2 shRNA or control shRNA were treated with increasing concentrations of erlotinib for 48 hours. Cell growth was determined by SRB assays. Data are mean±SD from 3 independent experiments.

FIG. 3 shows data indicating decreased PTPMeg2 and increased levels of pSTAT3 and Bcl2/Bcl-XL are associated with erlotinib resistance in human lung cancer cells. A, HCC827 cells and acquired erlotinib-resistant HCC827/ER cells were treated with increasing concentrations of erlotinib (Erlo) for 48 hours. Cell growth was analyzed by SRB assay. B, HCC827 and HCC827/ER cells were treated with erlotinib (Erlo, 1 μmol/L), and colony formation assay was conducted as described in Materials and Methods. C, pSTAT3, PTPMeg2, Bcl2, Bcl-XL, etc., were analyzed by Western Blot analysis. D, mRNA levels of Bcl2, Bcl-XL, and Mcl-1 were analyzed by RT-PCR.

FIG. 4 shows data indicating treatment of cells with niclosamide blocks erlotinib-induced activation of STAT3/Bcl2/Bcl-XL and reverses erlotinib resistance. A, HCC827 and HCC827/ER cells were treated with erlotinib (Erlo, 0.1 μmol/L) in the absence or presence of increasing concentrations of niclosamide (Niclo) for 48 hours. pSTAT3, Bcl2, Bcl-XL, and Mcl-1 were analyzed by Western Blot analysis. B, HCC827 and HCC827/ER cells were treated with erlotinib (Erlo), niclosamide (Niclo), or their combination for 48 hours. Cell growth was evaluated by SRB assay.

FIG. 5 shows data indicating specific depletion of STAT3 reverses erlotinib resistance. A, STAT3 shRNA or control shRNA was transfected into HCC827 cells. Expression levels of STAT3, Bcl-XL, and Bcl2 were analyzed by Western blot analysis. B, HCC827 cells expressing STAT3 shRNA or control shRNA were treated with erlotinib (Erlo) for 48 hours. Cell growth was determined by SRB assays.

FIG. 6 shows data indicating a combination of erlotinib and niclosamide overcomes acquired erlotinib resistance in vivo. A, mice bearing HCC827 or HCC827/ER lung cancer xenografts were treated with vehicle control, erlotinib (Erlo, 40 mg/kg/d), niclosamide (Niclo, 20 mg/kg/d), or their combination for 32 days. Each group includes 8 mice. Tumor volume was measured once every 2 days. After 32 days, the mice were sacrificed and the tumors were removed and analyzed. Representative tumor pictures were taken. B, active caspase-3 was analyzed in tumor tissues at the end of experiments by IHC staining and quantified as described in Materials and Methods. C, expression levels of pEGFR, pSTAT3, Bcl2, and Bcl-XL from tumor tissues in various treatment groups were analyzed by Western blot analysis.

FIG. 7 shows data indicating a specific depletion of STAT3 sensitizes head and neck cancer cells to erlotinib. A, STAT3 shRNA or control shRNA was transfected into Tu212 and Tu686 cells. Expression levels of STAT3, Bcl-XL and Bcl2 were analyzed by Western blot. B, Tu212 and Tu686 cells expressing STAT3 shRNA or control shRNA were treated with erlotinib for 48 h. Cell growth was determined by SRB assays.

FIG. 8 shows data indicating inhibition of STAT3 by niclosamide blocks erlotinib-induced STAT3 phosphorylation and sensitizes head and neck cancer cells to erlotinib. A, Tu212 and Tu686 cells were treated with erlotinib in the absence or presence of increasing concentrations of niclosamide for 48 h. pSTAT3, Bcl2 and Bcl-XL were analyzed by Western Blot. B, Tu212 and Tu686 cells were treated with erlonitib, niclosamide or their combination. After 48 h, cell growth was determined by SRB assays. Combination index (CI) values were calculated.

FIG. 9 shows data indicating the combination of erlotinib and niclosamide synergistically represses head and neck cancer growth in vivo. A, Mice bearing Tu212 xenografts were treated with vehicle control, erlotinib (Erlo, 40 mg/kg/d), niclosamide (Niclo, 20 mg/kg/d) or their combination for 14 days. Each group included 8 mice. Tumor volume was measured once every 2 days. After 14 days, the mice were sacrificed and the tumors were removed and analyzed. Representative tumor pictures were taken. B, Active caspase 3 and Ki 67 were analyzed in tumor tissues at the end of experiments by IHC staining and quantified. C, Expression levels of pEGFR, pSTAT3, Bcl2 and Bcl-XL in tumor tissues from various treatment groups were analyzed by Western blot.

FIG. 10 shows data indicating toxicity analysis in mice bearing Tu212 xenografts treated with erlotinib and niclosamide. A, Body weight of mice with Tu212 xenografts was measured once every other day during treatment with vehicle control, erlotinib (Erlo, 40 mg/kg/d), niclosamide (Niclo, 20 mg/kg/d) or their combination. B, Blood analysis of mice after various treatments for 14 days. C, H&E histology of various organs after treatments.

### DETAILED DISCUSSION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, physiology, and the like, which are within the skill of the art. Such techniques are explained fully in the literature.

It must be noted that, as used in the specification and the appended claims, the singular forms “a,” “an,” and “the” include plural referents unless the context clearly dictates otherwise. In this specification and in the claims that follow, reference will be made to a number of terms that shall be defined to have the following meanings unless a contrary intention is apparent.

A “subject” refers to a human, animal, laboratory animal, livestock, or domestic pet.

As used herein, “salts” refer to derivatives of the disclosed compounds where the parent compound is modified making acid or base salts thereof. Examples of salts include, but are not limited to, mineral or organic acid salts of basic residues such as amines, alkylamines, or dialkylamines; alkali or organic salts of acidic residues such as carboxylic acids; and the like. In preferred embodiment the salts are conventional nontoxic pharmaceutically acceptable salts including the quaternary ammonium salts of the parent compound formed, and non-toxic inorganic or organic acids. Preferred salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

The term “prodrug” refers to an agent that is converted into a biologically active form in vivo. Prodrugs are often useful because, in some situations, they may be easier to administer than the parent compound. They may, for instance, be bioavailable by oral administration whereas the parent compound is not. The prodrug may also have improved solubility in pharmaceutical compositions over the parent drug. A prodrug may be converted into the parent drug by various mechanisms, including enzymatic processes and metabolic hydrolysis.

As used herein, the terms “treat” and “treating” are not limited to the case where the subject (e.g. patient) is cured and the disease is eradicated. Rather, embodiments, of the present disclosure also contemplate treatment that merely reduces symptoms, and/or delays disease progression.

As used herein, the term “combination with” when used to describe administration with an additional treatment means that the agent may be administered prior to, together with, or after the additional treatment, or a combination thereof.

As used herein, the term “derivative” refers to a structurally similar compound that retains sufficient functional attributes of the identified analogue. The derivative may be structurally similar because it is lacking one or more atoms, substituted, a salt, in different hydration/oxidation states, or because one or more atoms within the molecule are switched, such as, but not limited to, replacing a oxygen atom with a sulfur atom or replacing an amino group with a hydroxyl group. The derivative may be a prodrug. Derivatives may be prepare by any variety of synthetic methods or appropriate adaptations presented in synthetic or organic chemistry text books, such as those provide in March's Advanced Organic Chemistry: Reactions, Mechanisms, and Structure, Wiley, 6th Edition (2007) Michael B. Smith or Domino Reactions in Organic Synthesis, Wiley (2006) Lutz F. Tietze hereby incorporated by reference.

“Cancer” refers any of various cellular diseases with malignant neoplasms characterized by the proliferation of cells. It is not intended that the diseased cells must actually invade surrounding tissue and metastasize to new body sites. Cancer can involve any tissue of the body and have many different forms in each body area. Within the context of certain embodiments, whether “cancer is reduced” can be identified by a variety of diagnostic manners known to one skill in the art including, but not limited to, observation the reduction in size or number of tumor masses or if an increase of apoptosis of cancer cells observed, e.g., if more than a 5% increase in apoptosis of cancer cells is observed for a sample compound compared to a control without the compound. It can also be identified by a change in relevant biomarker or gene expression profile, such as PSA for prostate cancer, HER2 for breast cancer, or others.

Niclosamide Overcomes Acquired Resistance to Erlotinib through Suppression of STAT3 in Non-Small Cell Lung Cancer

It has been discovered that erlotinib enhances STAT3 phosphorylation by downregulation of its phosphatase PTPMeg2, which leads to elevated levels of Bcl2/Bcl-XL and consequent loss of erlotinib sensitivity. Niclosamide blocks erlotinib-induced activation of the STAT3/Bcl2/Bcl-XL survival pathway in lung cancer cells, leading to the reversal of erlotinib resistance in vitro and in vivo. Experiments herein also indicate combined inhibition of EGFR and STAT3 using erlotinib and niclosamide synergistically represses head and neck cancer in vitro and in vivo; thus is an effective therapeutic strategy for improving prognosis of patients with lung, head, and neck cancer.

The emergence of resistance to EGF receptor (EGFR) inhibitor therapy is a major clinical problem for patients with non-small cell lung cancer (NSCLC). The mechanisms underlying tumor resistance to inhibitors of the kinase activity of EGFR are not fully understood. Studies reported herein indicate that inhibition of EGFR by erlotinib induces STAT3 phosphorylation at Tyr705 in association with increased Bcl2/Bcl-XL at both mRNA and protein levels in various human lung cancer cells. PTPMeg2 is a physiologic STAT3 phosphatase that can directly dephosphorylate STAT3 at the Tyr705 site. Treatment of cells with erlotinib results in downregulation of PTPMeg2 without activation of STAT3 kinases [i.e., Janus-activated kinase (JAK2) or c-Src], suggesting that erlotinib-enhanced phosphorylation of STAT3 may occur, at least in part, from suppression of PTPMeg2 expression. Because elevated levels of phosphorylated STAT3 (pSTAT3), Bcl2, and Bcl-XL were observed in erlotinib-resistant lung cancer (HCC827/ER) cells as compared with erlotinib-sensitive parental HCC827 cells, it is proposed that the erlotinib-activated STAT3/Bcl2/Bcl-XL survival pathway may contribute to acquired resistance to erlotinib. Both blockage of Tyr705 phosphorylation of STAT3 by niclosamide and depletion of STAT3 by RNA interference in HCC827/ER cells reverse erlotinib resistance. Niclosamide in combination with erlotinib potently represses erlotinib-resistant lung cancer xenografts in association with increased apoptosis in tumor tissues, suggesting that niclosamide can restore sensitivity to erlotinib. These findings indicate a mechanism of erlotinib resistance and indicate that one approach to overcoming acquired resistance is by blocking the STAT3/Bcl2/Bcl-XL survival signaling pathway in human lung cancer.

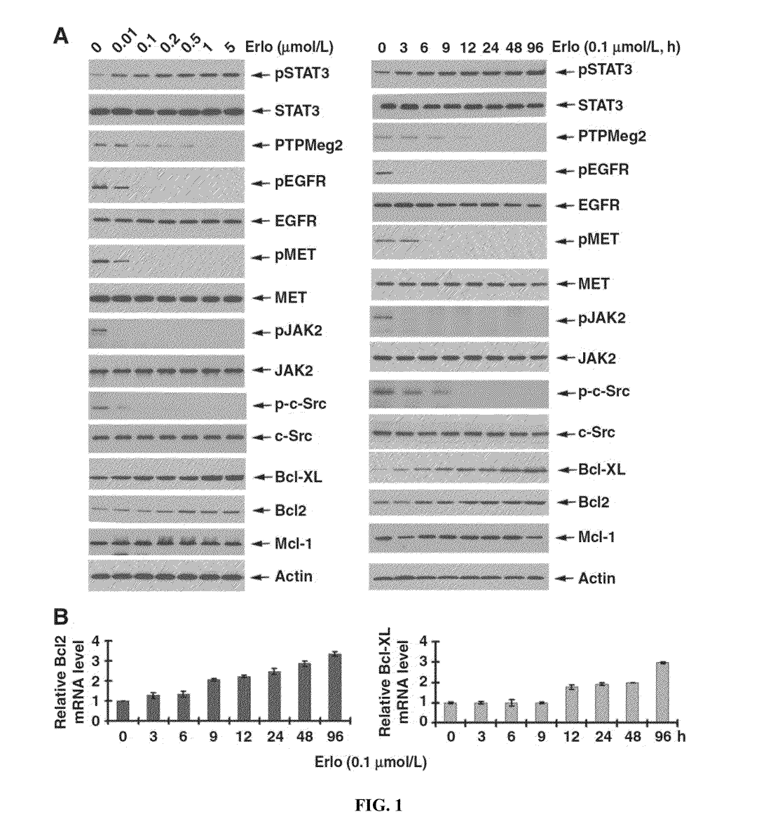

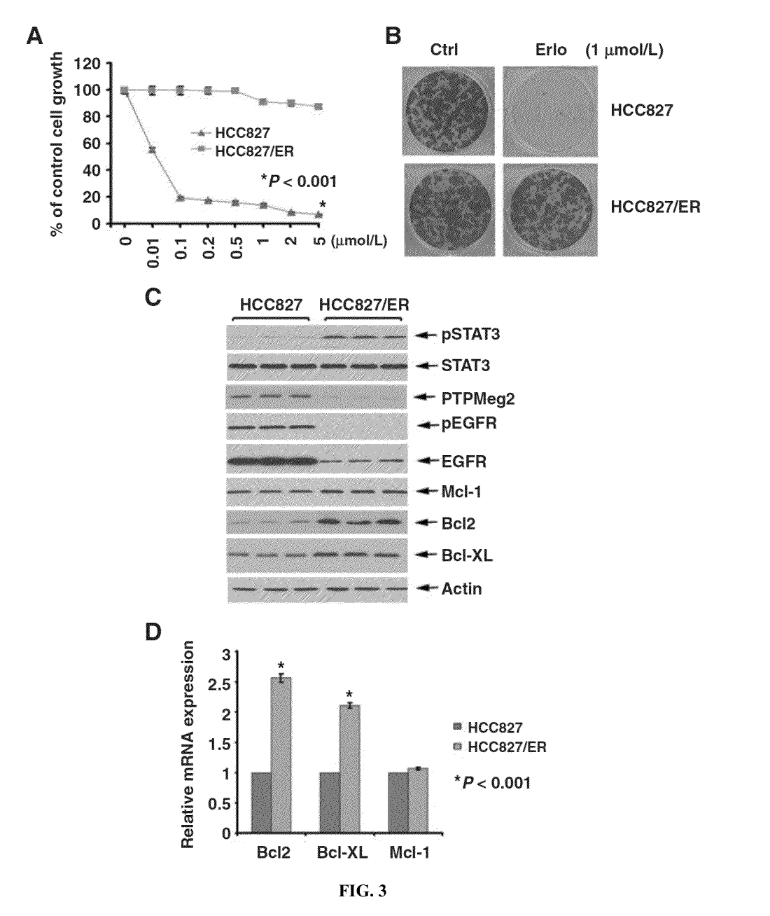

The EGFR TKIs, erlotinib and gefitinib, are effective agents for the treatment of patients with NSCLC whose tumors bear activating somatic mutations in EGFR gene (exon 19 deletions or exon 21 L858R substitution). The vast majority of patients with EGFR-mutant advanced NSCLCs will show an initial impressive tumor response to EGFR-TKIs. However, over a median period of approximately 12 months, resistance develops leading to a loss of treatment efficacy of the EGFR-TKIs. Two major mechanisms of resistance to EGFR-TKIs have been identified: (i) secondary resistance mutations and (ii) “oncogene kinase switch” (i.e., amplification of the MET oncogene). These 2 mechanisms (T790M mutation and MET amplification) account for 70% of all cases of acquired resistance to EGFR-TKIs. Other secondary resistance mutations (i.e., D761Y, L747S, and T854A) seem to be rare (<5%). Since “multiple” molecular mechanisms at play in “late resistance” besides T790M-EGFR and MET-amplification, identification of these mechanisms that mediate the remaining instances of otherwise unexplained acquired EGFR-TKI resistance is important for lung cancer therapy. It was discovered that inhibition of EGFR by erlotinib induces the activation of STAT3 by suppressing its physiologic phosphatase PPTMeg2 leading to increased Bcl2/Bcl-XL, which contributes to the acquired erlotinib resistance (FIGS. 1-3). Interestingly, Mcl-1 did not seem to be altered or impacted in the erlotinib resistance in this model system.

It is possible that early pSTAT3 activation induced by erlotinib in the early adaptive drug-resistant tumor cells may ultimately become late resistance by irreversibly maintaining activation of the pSTAT3/Bcl2/Bcl-xL survival pathway. Studies herein focus on irreversible “late resistance” rather than “early adaptive TKI resistance.” It is currently unclear how erlotinib stimulates STAT3 phosphorylation. Most studies have attributed increased STAT3 phosphorylation to overactivation of JAK or Src kinase. As shown in FIG. 1, erlotinib inhibited JAK2 and c-Src phosphorylation. Thus, erlotinib-induced STAT3 phosphorylation does not likely result from JAK2 or c-Src in this case. STAT3 phosphorylation status is also tightly regulated by dephosphorylation through its physiologic phosphatase PTPMeg2. As erlotinib inhibited the physiologic STAT3 phosphatase PTPMeg2 (FIG. 1A), this could reduce its ability to dephosphorylate STAT3. Because reduced dephosphorylation could lead to elevated phosphorylation, erlotinib-induced STAT3 phosphorylation may occur, at least in part, through suppression of its phosphatase PTPMeg2.

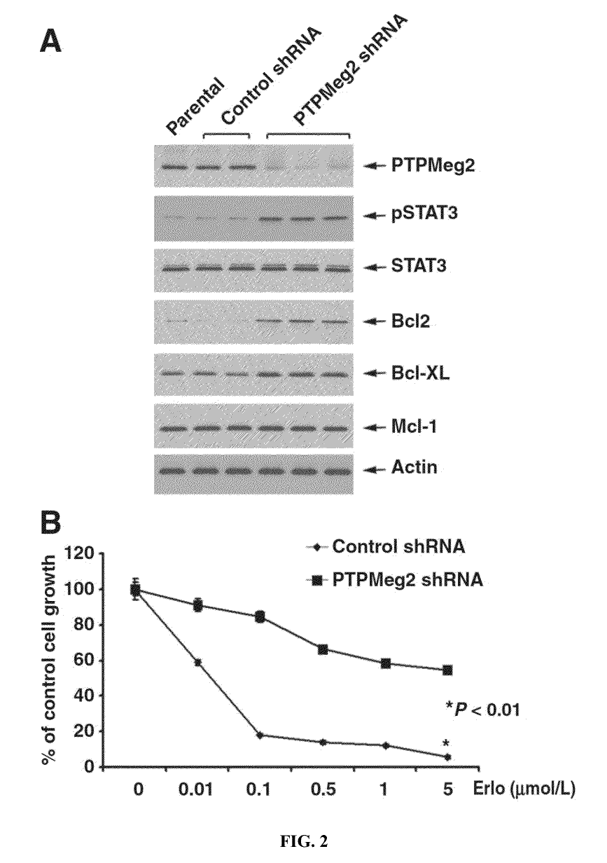

Selective depletion of PTPMeg2 activates the STAT3/Bcl2/Bcl-XL survival pathway that also contributes to decreased sensitivity to erlotinib (FIG. 2). Because decreased PTPMeg2 and increased pSTAT3/Bcl2/Bcl-XL were also observed in HCC827/ER cells with acquired erlotinib resistance (vs. parental HCC827), this indicates that erlotinib activation of STAT3/Bcl2/Bcl-XL by suppression PTPMeg2 contributes to acquired erlotinib resistance. It is important that other mechanisms of resistance such as acquired genetic mutations (i.e., T790M, T854A, L747S, and D761Y) were excluded by DNA sequencing.

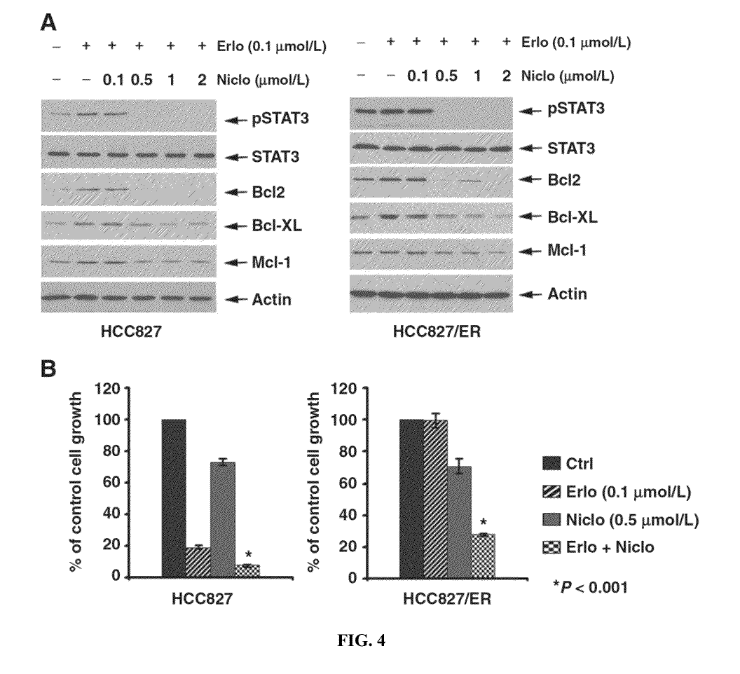

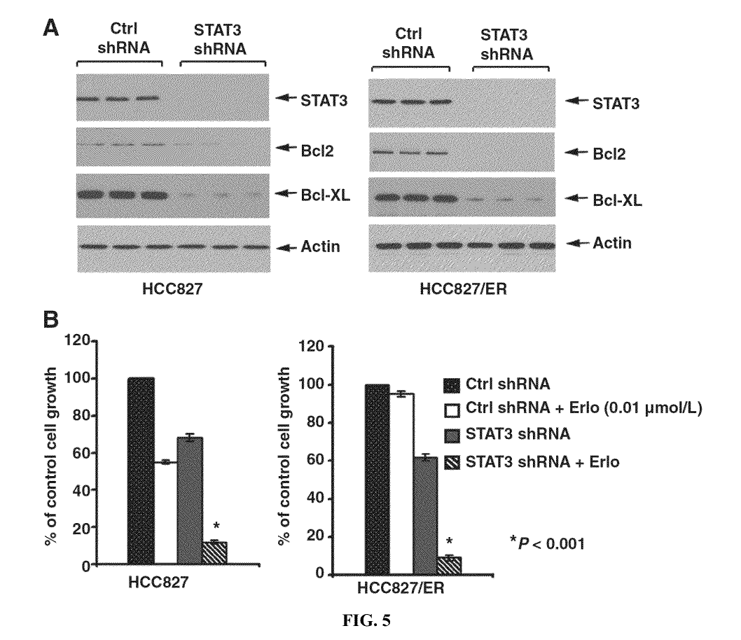

Niclosamide is an U.S. Food and Drug Administration-approved small-molecule drug of the teniacide anthelmintic family that is effective against human tapeworms. It exerts its anthelminthic effects by uncoupling oxidative phosphorylation in the tapeworm. Niclosamide is safe, well tolerated, inexpensive, and readily available. Niclosamide is a small-molecule STAT3 inhibitor because it can inhibit Tyr705 site phosphorylation as well as transcriptional activity of STAT3, but has no obvious inhibitory effect on the activation of the upstream proteins JAK2 and Src. Data herein reveal that niclosamide not only blocks erlotinib-induced STAT3 phosphorylation, but also suppresses its downstream effectors, Bcl2 and Bcl-XL, in both HCC827 and HCC827/ER cells (FIG. 4A), indicating that niclosamide functions as a STAT3 inhibitor to block erlotinib-activated STAT3/Bcl2/Bcl-XL survival pathway in human lung cancer cells. Importantly, niclosamide can reverse erlotinib resistance and restore the sensitivity of HCC827/ER cells to erlotinib (FIG. 4B). Because specific depletion of STAT3 using RNAi sensitizes HCC827 cells to erlotinib and also overcomes erlotinib resistance in HCC827/ER cells through reduced Bcl2/Bcl-XL (FIG. 5), this indicates that the STAT3/Bcl2/Bcl-XL survival pathway is important for optimal efficacy and the development of resistance to erlotinib therapy.

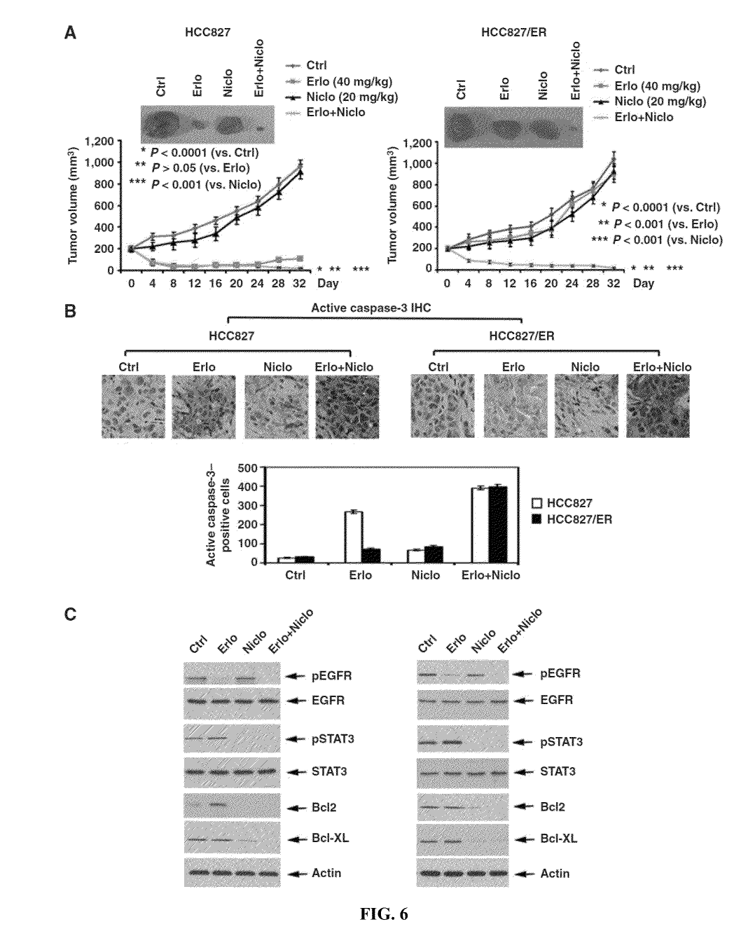

To further evaluate whether blockage of the STAT3/Bcl2/Bcl-XL survival pathway by niclosamide overcomes erlotinib resistance in vivo, erlotinib-sensitive (i.e., HCC827) and erlotinib-resistant (HCC827/ER) xenografts were compared with various treatments as shown in FIG. 6. Niclosamide (20 mg/kg/d) in combination with a dose of erlotinib (40 mg/kg/d) effectively overcame erlotinib resistance in HCC827/ER xenografts (FIG. 6). In certain embodiments, the disclosure contemplates administering Niclosamide at (10-30 or 5-40 mg/kg/d) and erlotinid at (20-30 or 10-40 mg/kg/d). The long-term tumor regression seen in some of the mice treated with the combination of erlotinib and niclosamide suggests that this combination is of clinical translation in human subjects with lung cancer.

### Combination Therapies

The cancer treatments disclosed herein can be applied as a sole therapy or can involve, conventional surgery or radiotherapy or chemotherapy. Such chemotherapy can include one or more of the following categories of anti-tumor agents:

(i) antiproliferative/antineoplastic drugs and combinations thereof, as used in medical oncology, such as alkylating agents (for example cis-platin, carboplatin, cyclophosphamide, nitrogen mustard, melphalan, chlorambucil, busulfan and nitrosoureas); antimetabolites (for example antifolates such as fluoropyrimidines like 5-fluorouracil and gemcitabine, tegafur, raltitrexed, methotrexate, cytosine arabinoside and hydroxyurea); antitumor antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere); and topoisomerase inhibitors (for example epipodophyllotoxins like etoposide and teniposide, amsacrine, topotecan and camptothecin); and proteosome inhibitors (for example bortezomib [Velcade®]); and the agent anegrilide [Agrylin®]; and the agent alpha-interferon

(ii) cytostatic agents such as antioestrogens (for example tamoxifen, toremifene, raloxifene, droloxifene and iodoxyfene), oestrogen receptor down regulators (for example fulvestrant), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride;

(iii) agents which inhibit cancer cell invasion (for example metalloproteinase inhibitors like marimastat and inhibitors of urokinase plasminogen activator receptor function);

(iv) inhibitors of growth factor function, for example such inhibitors include growth factor antibodies, growth factor receptor antibodies (for example the anti-Her2 antibody trastuzumab and the anti-epidermal growth factor receptor (EGFR) antibody, cetuximab), farnesyl transferase inhibitors, tyrosine kinase inhibitors and serine/threonine kinase inhibitors, for example inhibitors of the epidermal growth factor family for example EGFR family tyrosine kinase inhibitors such as: N-(3-chloro-4-fluorophenyl)-7-methoxy-6-(3-morpholinopropoxy)quinazolin-4-amine (gefitinib), N-(3-ethynylphenyl)-6,7-bis(2-methoxyethoxy)quinazolin-4-amine (erlotinib), and 6-acrylamido-N-(3-chloro-4-fluorophenyl)-7-(3-morpholinopropoxy)quinazolin-4-amine (CI 1033), for example inhibitors of the platelet-derived growth factor family and for example inhibitors of the hepatocyte growth factor family, for example inhibitors of phosphotidylinositol 3-kinase (PI3K) and for example inhibitors of mitogen activated protein kinase kinase (MEK1/2) and for example inhibitors of protein kinase B (PKB/Akt), for example inhibitors of Src tyrosine kinase family and/or Abelson (AbI) tyrosine kinase family such as dasatinib (BMS -354825) and imatinib mesylate (Gleevec™); and any agents that modify STAT signalling;

(v) antiangiogenic agents such as those which inhibit the effects of vascular endothelial growth factor, (for example the anti-vascular endothelial cell growth factor antibody bevacizumab [Avastin™]) and compounds that work by other mechanisms (for example linomide, inhibitors of integrin ocvβ3 function and angiostatin);

(vi) vascular damaging agents such as Combretastatin A4;

(vii) antisense therapies, for example those which are directed to the targets listed above, such as an anti-RAS antisense; and

(viii) immunotherapy approaches, including for example ex-vivo and in-vivo approaches to increase the immunogenicity of patient tumor cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumor cell lines and approaches using anti-idiotypic antibodies, and approaches using the immunomodulatory drugs thalidomide and lenalidomide [Revlimid®].

### Formulations

Pharmaceutical compositions disclosed herein can be in the form of pharmaceutically acceptable salts, as generally described below. Some preferred, but non-limiting examples of suitable pharmaceutically acceptable organic and/or inorganic acids are hydrochloric acid, hydrobromic acid, sulfuric acid, nitric acid, acetic acid and citric acid, as well as other pharmaceutically acceptable acids known per se (for which reference is made to the references referred to below).

When the compounds of the disclosure contain an acidic group as well as a basic group, the compounds of the disclosure can also form internal salts, and such compounds are within the scope of the disclosure. When a compound contains a hydrogen-donating heteroatom (e.g. NH), salts are contemplated to cover isomers formed by transfer of the hydrogen atom to a basic group or atom within the molecule.

Pharmaceutically acceptable salts of the compounds include the acid addition and base salts thereof. Suitable acid addition salts are formed from acids which form non-toxic salts. Examples include the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts. Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts. Hemisalts of acids and bases can also be formed, for example, hemisulphate and hemicalcium salts. For a review on suitable salts, see Handbook of Pharmaceutical Salts: Properties, Selection, and Use by Stahl and Wermuth (Wiley-VCH, 2002), incorporated herein by reference.

The compounds described herein can be administered in the form of prodrugs. A prodrug can include a covalently bonded carrier which releases the active parent drug when administered to a mammalian subject. Prodrugs can be prepared by modifying functional groups present in the compounds in such a way that the modifications are cleaved, either in routine manipulation or in vivo, to the parent compounds. Prodrugs include, for example, compounds wherein a hydroxyl group is bonded to any group that, when administered to a mammalian subject, cleaves to form a free hydroxyl group. Examples of prodrugs include, but are not limited to, acetate, formate and benzoate derivatives of alcohol functional groups in the compounds. Examples of structuring a compound as prodrugs can be found in the book of Testa and Caner, Hydrolysis in Drug and Prodrug Metabolism, Wiley (2006) hereby incorporated by reference. Typical prodrugs form the active metabolite by transformation of the prodrug by hydrolytic enzymes, the hydrolysis of amides, lactams, peptides, carboxylic acid esters, epoxides or the cleavage of esters of inorganic acids.

Pharmaceutical compositions typically comprise an effective amount of a compound and a suitable pharmaceutical acceptable carrier. The preparations can be prepared in a manner known per se, which usually involves mixing the at least one compound according to the disclosure with the one or more pharmaceutically acceptable carriers, and, if desired, in combination with other pharmaceutical active compounds, when necessary under aseptic conditions. Reference is made to U.S. Pat. No. 6,372,778, U.S. Pat. No. 6,369,086, U.S. Pat. No. 6,369,087 and U.S. Pat. No. 6,372,733 and the further references mentioned above, as well as to the standard handbooks, such as the latest edition of Remington's Pharmaceutical Sciences. It is well known that ester prodrugs are readily degraded in the body to release the corresponding alcohol. See e.g., Imai, Drug Metab Pharmacokinet. (2006) 21(3):173-85, entitled “Human carboxylesterase isozymes: catalytic properties and rational drug design.”

Generally, for pharmaceutical use, the compounds can be formulated as a pharmaceutical preparation comprising at least one compound and at least one pharmaceutically acceptable carrier, diluent or excipient and/or adjuvant, and optionally one or more further pharmaceutically active compounds.

The pharmaceutical preparations of the disclosure are preferably in a unit dosage form, and can be suitably packaged, for example in a box, blister, vial, bottle, sachet, ampoule or in any other suitable single-dose or multi-dose holder or container (which can be properly labeled); optionally with one or more leaflets containing product information and/or instructions for use. Generally, such unit dosages will contain between 1 and 1000 mg, and usually between 5 and 500 mg, of the at least one compound of the disclosure e.g., about 10, 25, 50, 100, 200, 300 or 400 mg per unit dosage.

The compounds can be administered by a variety of routes including the oral, ocular, rectal, transdermal, subcutaneous, intravenous, intramuscular or intranasal routes, depending mainly on the specific preparation used. The compound will generally be administered in an “effective amount,” by which it is meant any amount of a compound that, upon suitable administration, is sufficient to achieve the desired therapeutic or prophylactic effect in the subject to which it is administered. Usually, depending on the condition to be prevented or treated and the route of administration, such an effective amount will usually be between 0.01 to 1000 mg per kilogram body weight of the patient per day, more often between 0.1 and 500 mg, such as between 1 and 250 mg, for example about 5, 10, 20, 50, 100, 150, 200 or 250 mg, per kilogram body weight of the patient per day, which can be administered as a single daily dose, divided over one or more daily doses. The amount(s) to be administered, the route of administration and the further treatment regimen can be determined by the treating clinician, depending on factors such as the age, gender and general condition of the patient and the nature and severity of the disease/symptoms to be treated. Reference is made to U.S. Pat. No. 6,372,778, U.S. Pat. No. 6,369,086, U.S. Pat. No. 6,369,087 and U.S. Pat. No. 6,372,733 and the further references mentioned above, as well as to the standard handbooks, such as the latest edition of Remington's Pharmaceutical Sciences.

Formulations containing one or more of the compounds described herein can be prepared using a pharmaceutically acceptable carrier composed of materials that are considered safe and effective and can be administered to an individual without causing undesirable biological side effects or unwanted interactions. The carrier is all components present in the pharmaceutical formulation other than the active ingredient or ingredients. As generally used herein “carrier” includes, but is not limited to, diluents, binders, lubricants, disintegrators, fillers, pH modifying agents, preservatives, antioxidants, solubility enhancers, and coating compositions.

Carrier also includes all components of the coating composition which can include plasticizers, pigments, colorants, stabilizing agents, and glidants. Delayed release, extended release, and/or pulsatile release dosage formulations can be prepared as described in standard references such as “Pharmaceutical dosage form tablets,” eds. Liberman et. al. (New York, Marcel Dekker, Inc., 1989), “Remington—The science and practice of pharmacy,” 20th ed., Lippincott Williams & Wilkins, Baltimore, Md., 2000, and “Pharmaceutical dosage forms and drug delivery systems,” 6th Edition, Ansel et al., (Media, PA: Williams and Wilkins, 1995). These references provide information on carriers, materials, equipment and process for preparing tablets and capsules and delayed release dosage forms of tablets, capsules, and granules.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Additionally, the coating material can contain conventional carriers such as plasticizers, pigments, colorants, glidants, stabilization agents, pore formers and surfactants.

Optional pharmaceutically acceptable excipients present in the drug-containing tablets, beads, granules or particles include, but are not limited to, diluents, binders, lubricants, disintegrants, colorants, stabilizers, and surfactants.

Diluents, also referred to as “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar.

Binders are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

Lubricants are used to facilitate tablet manufacture. Examples of suitable lubricants include, but are not limited to, magnesium stearate, calcium stearate, stearic acid, glycerol behenate, polyethylene glycol, talc, and mineral oil.

Disintegrants are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (Polyplasdone XL from GAF Chemical Corp).

Stabilizers are used to inhibit or retard drug decomposition reactions which include, by way of example, oxidative reactions.

Surfactants can be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-.beta.-alanine, sodium N-lauryl-.beta.-iminodipropionate, myristoamphoacetate, lauryl betaine and lauryl sulfobetaine.

If desired, the tablets, beads, granules, or particles can also contain minor amount of nontoxic auxiliary substances such as wetting or emulsifying agents, dyes, pH buffering agents, or preservatives.

The compositions described herein can be formulation for modified or controlled release. Examples of controlled release dosage forms include extended release dosage forms, delayed release dosage forms, pulsatile release dosage forms, and combinations thereof.

The extended release formulations are generally prepared as diffusion or osmotic systems, for example, as described in “Remington—The science and practice of pharmacy” (20th ed., Lippincott Williams & Wilkins, Baltimore, Md., 2000). A diffusion system typically consists of two types of devices, a reservoir and a matrix, and is well known and described in the art. The matrix devices are generally prepared by compressing the drug with a slowly dissolving polymer carrier into a tablet form. The three major types of materials used in the preparation of matrix devices are insoluble plastics, hydrophilic polymers, and fatty compounds. Plastic matrices include, but are not limited to, methyl acrylate-methyl methacrylate, polyvinyl chloride, and polyethylene. Hydrophilic polymers include, but are not limited to, cellulosic polymers such as methyl and ethyl cellulose, hydroxyalkylcelluloses such as hydroxypropyl-cellulose, hydroxypropylmethylcellulose, sodium carboxymethylcellulose, and Carbopol® 934, polyethylene oxides and mixtures thereof. Fatty compounds include, but are not limited to, various waxes such as carnauba wax and glyceryl tristearate and wax-type substances including hydrogenated castor oil or hydrogenated vegetable oil, or mixtures thereof.

In certain preferred embodiments, the plastic material is a pharmaceutically acceptable acrylic polymer, including but not limited to, acrylic acid and methacrylic acid copolymers, methyl methacrylate, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamine copolymer poly(methyl methacrylate), poly(methacrylic acid)(anhydride), polymethacrylate, polyacrylamide, poly(methacrylic acid anhydride), and glycidyl methacrylate copolymers.

In certain preferred embodiments, the acrylic polymer is comprised of one or more ammonio methacrylate copolymers Ammonio methacrylate copolymers are well known in the art, and are described in NF XVII as fully polymerized copolymers of acrylic and methacrylic acid esters with a low content of quaternary ammonium groups.

In one preferred embodiment, the acrylic polymer is an acrylic resin lacquer such as that which is commercially available from Rohm Pharma under the tradename Eudragit®. In further preferred embodiments, the acrylic polymer comprises a mixture of two acrylic resin lacquers commercially available from Rohm Pharma under the tradenames Eudragit® RL30D and Eudragit ® RS30D, respectively. Eudragit® RL30D and Eudragit® RS30D are copolymers of acrylic and methacrylic esters with a low content of quaternary ammonium groups, the molar ratio of ammonium groups to the remaining neutral (meth)acrylic esters being 1:20 in Eudragit® RL30D and 1:40 in Eudragit® RS30D. The mean molecular weight is about 150,000. Edragit® S-100 and Eudragit® L-100 are also preferred. The code designations RL (high permeability) and RS (low permeability) refer to the permeability properties of these agents. Eudragit® RL/RS mixtures are insoluble in water and in digestive fluids. However, multiparticulate systems formed to include the same are swellable and permeable in aqueous solutions and digestive fluids.

The polymers described above such as Eudragit® RL/RS can be mixed together in any desired ratio in order to ultimately obtain a sustained-release formulation having a desirable dissolution profile. Desirable sustained-release multiparticulate systems can be obtained, for instance, from 100% Eudragit® RL, 50% Eudragit® RL and 50% Eudragit® RS, and 10% Eudragit® RL and 90% Eudragit® RS. One skilled in the art will recognize that other acrylic polymers can also be used, such as, for example, Eudragit® L.

Alternatively, extended release formulations can be prepared using osmotic systems or by applying a semi-permeable coating to the dosage form. In the latter case, the desired drug release profile can be achieved by combining low permeable and high permeable coating materials in suitable proportion.

The devices with different drug release mechanisms described above can be combined in a final dosage form comprising single or multiple units. Examples of multiple units include, but are not limited to, multilayer tablets and capsules containing tablets, beads, or granules An immediate release portion can be added to the extended release system by means of either applying an immediate release layer on top of the extended release core using a coating or compression process or in a multiple unit system such as a capsule containing extended and immediate release beads.

Extended release tablets containing hydrophilic polymers are prepared by techniques commonly known in the art such as direct compression, wet granulation, or dry granulation. Their formulations usually incorporate polymers, diluents, binders, and lubricants as well as the active pharmaceutical ingredient. The usual diluents include inert powdered substances such as starches, powdered cellulose, especially crystalline and microcrystalline cellulose, sugars such as fructose, mannitol and sucrose, grain flours and similar edible powders. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders include substances such as starch, gelatin and sugars such as lactose, fructose, and glucose. Natural and synthetic gums, including acacia, alginates, methylcellulose, and polyvinylpyrrolidone can also be used. Polyethylene glycol, hydrophilic polymers, ethylcellulose and waxes can also serve as binders. A lubricant is necessary in a tablet formulation to prevent the tablet and punches from sticking in the die. The lubricant is chosen from such slippery solids as talc, magnesium and calcium stearate, stearic acid and hydrogenated vegetable oils.

Extended release tablets containing wax materials are generally prepared using methods known in the art such as a direct blend method, a congealing method, and an aqueous dispersion method. In the congealing method, the drug is mixed with a wax material and either spray-congealed or congealed and screened and processed.

Delayed release formulations are created by coating a solid dosage form with a polymer film, which is insoluble in the acidic environment of the stomach, and soluble in the neutral environment of the small intestine.

The delayed release dosage units can be prepared, for example, by coating a drug or a drug-containing composition with a selected coating material. The drug-containing composition can be, e.g., a tablet for incorporation into a capsule, a tablet for use as an inner core in a “coated core” dosage form, or a plurality of drug-containing beads, particles or granules, for incorporation into either a tablet or capsule. Preferred coating materials include bioerodible, gradually hydrolyzable, gradually water-soluble, and/or enzymatically degradable polymers, and can be conventional “enteric” polymers. Enteric polymers, as will be appreciated by those skilled in the art, become soluble in the higher pH environment of the lower gastrointestinal tract or slowly erode as the dosage form passes through the gastrointestinal tract, while enzymatically degradable polymers are degraded by bacterial enzymes present in the lower gastrointestinal tract, particularly in the colon. Suitable coating materials for effecting delayed release include, but are not limited to, cellulosic polymers such as hydroxypropyl cellulose, hydroxyethyl cellulose, hydroxymethyl cellulose, hydroxypropyl methyl cellulose, hydroxypropyl methyl cellulose acetate succinate, hydroxypropylmethyl cellulose phthalate, methylcellulose, ethyl cellulose, cellulose acetate, cellulose acetate phthalate, cellulose acetate trimellitate and carboxymethylcellulose sodium; acrylic acid polymers and copolymers, preferably formed from acrylic acid, methacrylic acid, methyl acrylate, ethyl acrylate, methyl methacrylate and/or ethyl methacrylate, and other methacrylic resins that are commercially available under the tradename Eudragit® (Rohm Pharma; Westerstadt, Germany), including Eudragit® L30D-55 and L100-55 (soluble at pH 5.5 and above), Eudragit® L-100 (soluble at pH 6.0 and above), Eudragit® S (soluble at pH 7.0 and above, as a result of a higher degree of esterification), and Eudragits® NE, RL and RS (water-insoluble polymers having different degrees of permeability and expandability); vinyl polymers and copolymers such as polyvinyl pyrrolidone, vinyl acetate, vinylacetate phthalate, vinylacetate crotonic acid copolymer, and ethylene-vinyl acetate copolymer; enzymatically degradable polymers such as azo polymers, pectin, chitosan, amylose and guar gum; zein and shellac. Combinations of different coating materials can also be used. Multi-layer coatings using different polymers can also be applied.

The preferred coating weights for particular coating materials can be readily determined by those skilled in the art by evaluating individual release profiles for tablets, beads and granules prepared with different quantities of various coating materials. It is the combination of materials, method and form of application that produce the desired release characteristics, which one can determine only from the clinical studies.

The coating composition can include conventional additives, such as plasticizers, pigments, colorants, stabilizing agents, glidants, etc. A plasticizer is normally present to reduce the fragility of the coating, and will generally represent about 10 wt. % to 50 wt. % relative to the dry weight of the polymer. Examples of typical plasticizers include polyethylene glycol, propylene glycol, triacetin, dimethyl phthalate, diethyl phthalate, dibutyl phthalate, dibutyl sebacate, triethyl citrate, tributyl citrate, triethyl acetyl citrate, castor oil and acetylated monoglycerides. A stabilizing agent is preferably used to stabilize particles in the dispersion. Typical stabilizing agents are nonionic emulsifiers such as sorbitan esters, polysorbates and polyvinylpyrrolidone. Glidants are recommended to reduce sticking effects during film formation and drying, and will generally represent approximately 25 wt. % to 100 wt. % of the polymer weight in the coating solution. One effective glidant is talc. Other glidants such as magnesium stearate and glycerol monostearates can also be used. Pigments such as titanium dioxide can also be used. Small quantities of an anti-foaming agent, such as a silicone (e.g., simethicone), can also be added to the coating composition.

Alternatively, each dosage unit in the capsule can comprise a plurality of drug-containing beads, granules or particles. As is known in the art, drug-containing “beads” refer to beads made with drug and one or more excipients or polymers. Drug-containing beads can be produced by applying drug to an inert support, e.g., inert sugar beads coated with drug or by creating a “core” comprising both drug and one or more excipients. As is also known, drug-containing “granules” and “particles” comprise drug particles that can or cannot include one or more additional excipients or polymers. In contrast to drug-containing beads, granules and particles do not contain an inert support. Granules generally comprise drug particles and require further processing. Generally, particles are smaller than granules, and are not further processed. Although beads, granules and particles can be formulated to provide immediate release, beads and granules are generally employed to provide delayed release.

### EXAMPLES

Niclosamide Overcomes Acquired Erlotinib Resistance in vivo Leading to Long-Term Tumor-Free Survival

To test whether niclosamide overcomes the acquired erlotinib resistance of lung cancer in vivo, xenografts derived from HCC827 cells or HCC827/ER cell lines were treated in groups of 8 mice with erlotinib (40 mg/kg/d), niclosamide (20 mg/kg/d), or their combination for 32 days as described in Materials and Methods. The control group was treated with 0.5% DMSO vehicle. Lung cancer xenografts derived from HCC827 cells were very sensitive to erlotinib treatment with significant tumor shrinkage, whereas xenografts derived from HCC827/ER cells were resistant to erlotinib treatment with tumor volumes comparable with those in the vehicle-treated control group (FIG. 6A). In contrast, combination of niclosamide and erlotinib significantly repressed lung tumors derived from erlotinib-resistant HCC827/ER cells, indicating that niclosamide can reverse acquired erlotinib resistance and restore sensitivity of HCC827/ER xenografts to erlotinib treatment in vivo (FIG. 6A). Intriguingly, 2 of the 8 HCC827/ER xenograft-bearing mice treated with combined erlotinib and niclosamide showed complete tumor regression (more than 8 months from the end of treatment). To determine whether erlotinib alone or in combination with niclosamide represses lung cancer via apoptosis in vivo, representative samples from harvested tumor tissues were analyzed by immunohistochemistry for active caspase-3. Results revealed that erlotinib enhances active caspase-3-positive cells only in HCC827 tumor tissues but not in HCC827/ER tumor tissues (FIG. 6B). In contrast, the combination of the 2 drugs increased active caspase-3-positive cells in both HCC827 and HCC827/ER tumor tissues (FIG. 6B). Western blot analysis of tumor lysates revealed that treatment of HCC827 and HCC827/ER mice with niclosamide also blocks STAT3 phosphorylation in association with reduced Bcl2 and Bcl-XL (FIG. 6C).

### Disruption of STAT3 Sensitizes Head and Neck Cancer Cells to Erlotinib

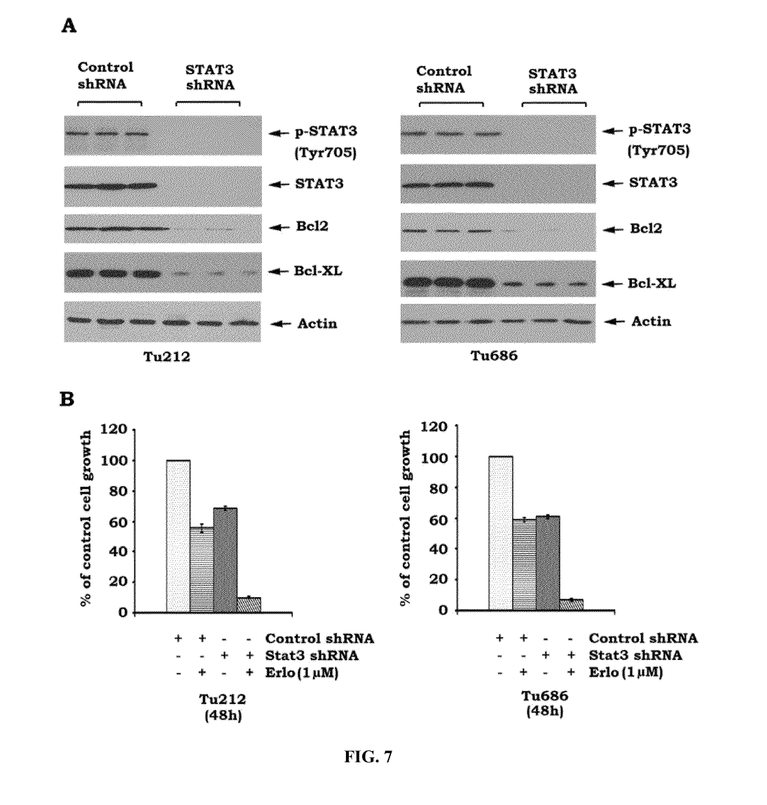

To test whether erlotinib-mediated STAT3 activation negatively affects erlotinib activity against head and neck cancer, STAT3 was knocked down from Tu212 and Tu686 cells using STAT3 shRNA (FIG. 7A). Silencing of STAT3 not only downregulates Bcl2 and Bcl-XL (FIG. 7A) but also significantly sensitizes head and neck cancer cells to erlotinib (FIG. 7B), suggesting that targeting STAT3 may have great potential to improve the efficacy of erlotinib against head and neck cancer.

Niclosamide Blocks Erlotinib-Induced STAT3 Phosphorylation and Synergizes with Erlotinib in the Suppression of Head and Neck Cancer Cell Growth

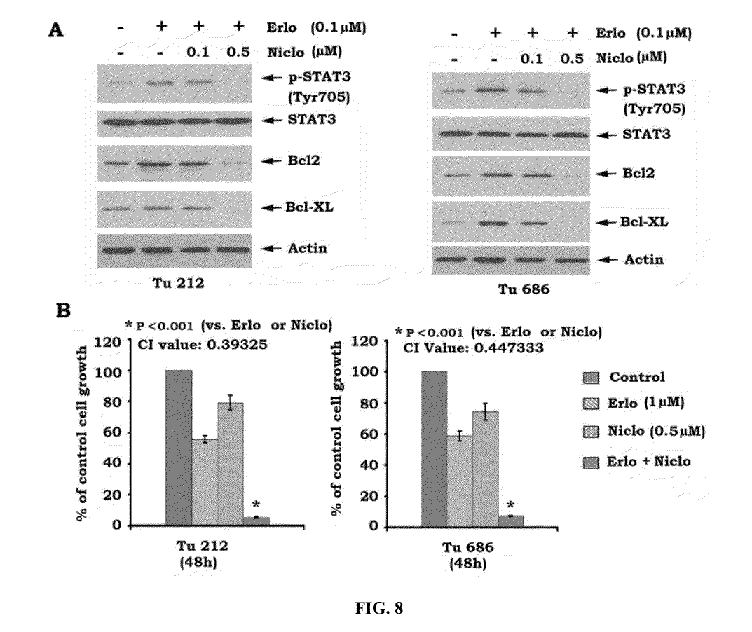

To test whether pharmacological disruption of STAT3 activity sensitizes head and neck cancer cells to erlotinib, Tu212 and Tu686 cells were treated with erlotinib in the absence or presence of increasing concentrations of niclosamide. Western blot analysis showed that niclosamide inhibited erlotinib-induced STAT3 phosphorylation and downregulated Bcl2/Bcl-XL in a dose-dependent manner (FIG. 8A). Importantly, niclosamide in combination with erlotinib significantly augmented growth inhibition of head and neck cancer cells (i.e. Tu212 and Tu686) (FIG. 8B). To more accurately analyze the degree of synergy between niclosamide and erlotinib, combination index (CI) values were calculated. The CI values were 0.39325 for Tu212 and 0.447333 for Tu686 cells, respectively. The CI values of less than 1 indicate that niclosamide and erlotinib exhibit synergistic growth inhibition of head and neck cancer cells.

### Niclosamide and Erlotinib Synergistically Inhibit the Growth of Head and Neck Cancer Animal Xenografts

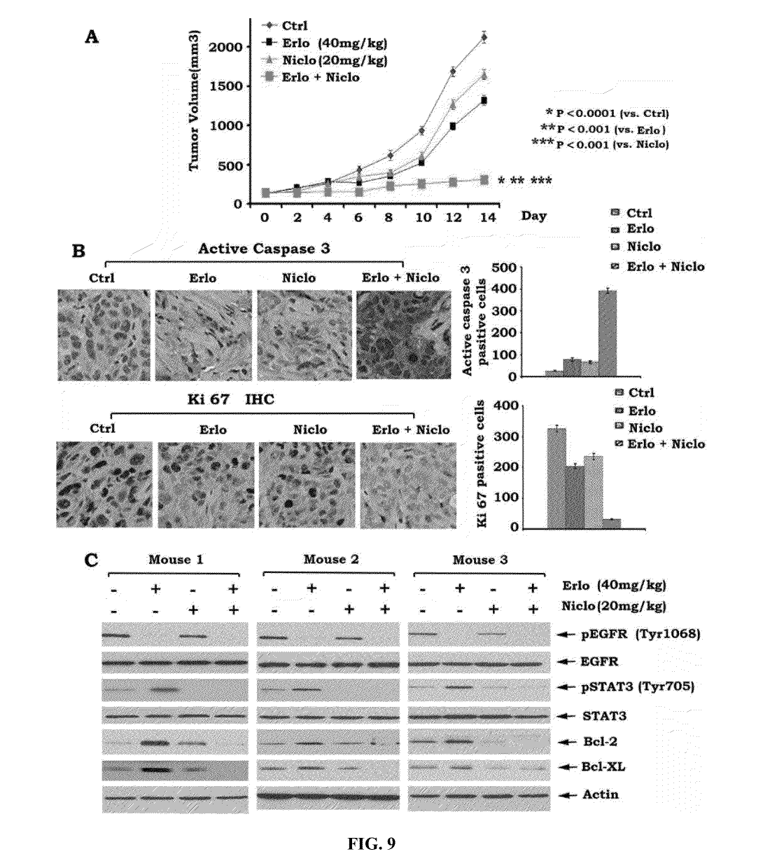

Since niclosamide and erlotinib play a synergistic role against head and neck cancer in vitro (FIG. 7), it is interesting to examine this synergistic effect in vivo. First, head and neck cancer xenografts were generated using Tu212 cells. Then, mice bearing Tu212 head and neck cancer xenografts were treated with erlotinib (40 mg/kg/d), niclosamide (20 mg/kg/d) alone or in combination for two weeks. Results showed that both erlotinib and niclosamide alone had modest efficacy against the xenograft tumors. However, the combination of erlotinib and niclosamide repressed xenograft tumor formation significantly more efficiently than either single agent alone in vivo (FIG. 9A). These data suggest that niclosamide and erlotinib have strong synergism in the treatment of head and neck cancer.

Apoptosis was measured by analysis of active caspase 3 in the tumor tissues by IHC staining The combination of erlotinib and niclosamide significantly increased active caspase 3 positive cells in association with decreased Ki-67 positive cells in head and neck tumor tissues (FIG. 9B). Protein analysis of tumor tissue lysates from three mice in each treatment group indicated that niclosamide blocks erlotinib-stimulated STAT3 phosphorylation leading to decreased Bcl2 and Bcl-XL levels (FIG. 9C). These data suggest the molecular mechanism by which niclosamide and erlotinib exhibit synergism against head and neck cancer growth in vivo.

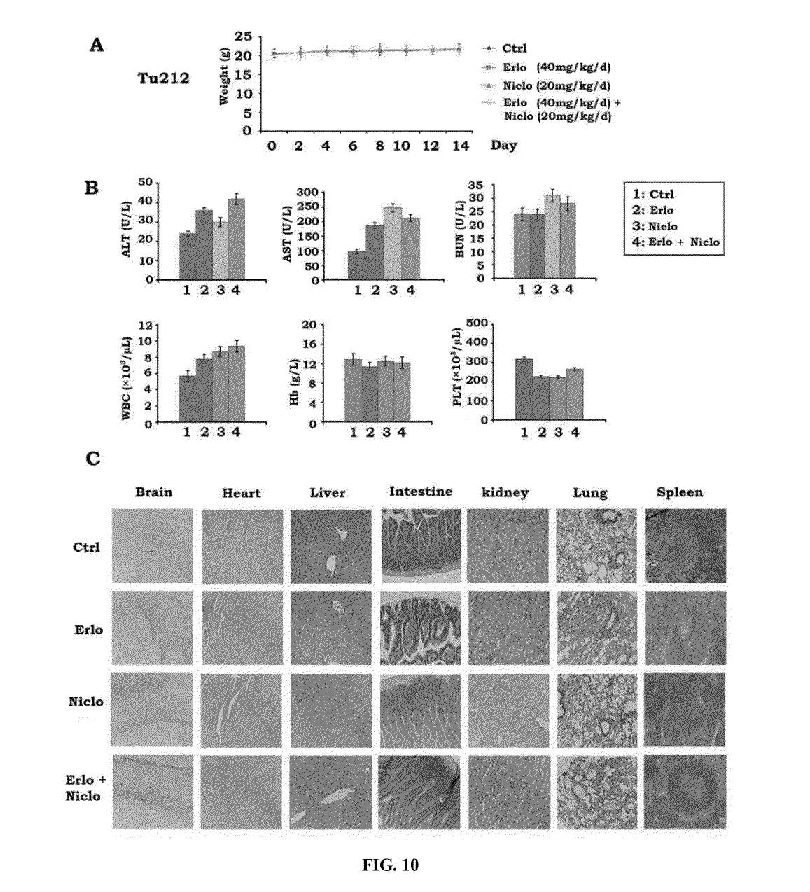

Importantly, treatments were well tolerated without weight loss during treatment (FIG. 10A). There were no observable alterations in vital organ functions as reflected by the results of liver, kidney, and bone marrow function tests (ALT, AST and BUN, WBC, Hb and platelet) (FIG. 10B). Histopathology of harvested normal tissues (brain, heart, lung, liver, spleen, kidney and intestine) revealed no evidence of toxicity in normal tissue (FIG. 10C).

1. A method of treating cancer comprising administering an effective amount of a STAT3 inhibitor in combinations with an EGFR tyrosine kinase inhibitor to a human subject in need thereof.

2. The method of claim 1, wherein the STAT3 inhibitor is niclosamide or salt thereof.

3. The method of claim 2, wherein the EGFR tyrosine kinase inhibitor is erlotinib or salt thereof.

4. The method of claim 3, wherein the subject is diagnosed with small cell lung cancer or non-small cell lung cancer.

5. The method of claim 4, wherein the subject is administered niclosamide at 100 to 3,000 mg per day.

6. The method of claim 4, wherein the subject is administered erlotinib at 100 to 200 mg per day.

7. The method of claim 3, wherein the subject is diagnosed with head or neck cancer.

8. The method of claim 7, wherein the subject is administered niclosamide at 100 to 3,000 mg per day.

9. The method of claim 7, wherein the subject is administered erlotinib at 100 to 200 mg per day.

10. A pharmaceutical compositions comprising niclosamide in combination with erlotinib and a pharmaceutically acceptable excipient.
